(19) United States
(12) Patent Application Publication
(10) Pub. No.: US 2013/0266118 A1
(43) Pub. Date: Oct. 10, 2013

(54) RADIATION IMAGING SYSTEM, CONTROL APPARATUS, AND CONTROL METHOD

(71) Applicant: CANON KABUSHIKI KAISHA, Tokyo (JP)

(72) Inventor: Daiya Senba, Inagi-shi (JP)

(73) Assignee: CANON KABUSHIKI KAISHA, Tokyo (JP)

(21) Appl. No.: 13/856,787

(22) Filed: Apr. 4, 2013

(30) Foreign Application Priority Data
Apr. 5, 2012 (JP) 2012-086504

(51) Int. Cl. A61B 6/00

(52) U.S. Cl. CPC A61B 6/545
USPC 378/62; 378/98

(57) ABSTRACT

A control apparatus for radiation imaging, the control apparatus includes an obtaining unit configured to obtain imaging information for performing radiation imaging; a display control unit configured to display a display screen for displaying the imaging information and a radiation image captured based on the imaging information; and a change destination obtaining unit configured to, with respect to one imaging information displayed on the display control unit, obtain imaging information about at least one change destination based on the imaging information, wherein the one imaging information displayed on the display control unit is changed to the specified imaging information among pieces of the obtained imaging information about at least one change destination.

### BACKGROUND OF THE INVENTION

1. Field of the Invention

The present disclosure relates to a radiation imaging system, a control apparatus, a control method, and a recording medium.

2. Description of the Related Art

Diagnosis and examination based on imaging using radiation (X-ray, for example) are actively performed. Such an examination advances based on an examination order including a plurality of imaging protocols.

The imaging protocol is manually produced by a radiological technician, for example, according to an examination order and includes an imaging region, an imaging method which are requested by a medical doctor and information about image processing.

Hospitals have gone online using a hospital information system (HIS) and a radiology information system (RIS). Along with these system, an examination order produced by the outside can be transferred to a radiation imaging apparatus via a network. In this case, an operator selects an imaging protocol from the examination order displayed on the display unit of the radiation imaging apparatus. According to the selection, a parameter is set based on the imaging protocol to perform imaging based on the parameter. If an incorrect imaging protocol is selected to perform imaging, a parameter is inappropriate, so that an intended imaging cannot be performed. In a medical examination, an image captured using a frontal chest imaging protocol sometimes needs to be processed by a parameter for pneumoconiosis. To cope with this case, there are known techniques discussed in Japanese Patent Applications Laid-Open No. 2009-254799 and No. 2011-50518.

In the technique discussed in Japanese Patent Application Laid-Open No. 2009-254799, an imaging protocol that has already used in imaging can be changed to another imaging protocol.

The captured image is different in the size of the image and an image processing method depending on the type of a radiation detector. Therefore, if the imaging protocol already used in imaging is desired to be changed to another imaging protocol, the imaging protocol to which the used imaging protocol is changed is limited to the imaging protocol of the same radiation detector.

However, the technique discussed in Japanese Patent Application Laid-Open No. 2009-254799 does not provide conditions for the imaging protocol which can be designated as the one to which the imaging protocol is changed. Therefore, in an imaging environment where a plurality of different types of radiation detectors is operated, an operator needs to search imaging protocols registered in the radiation imaging system for the imaging protocol of the same detector, so that it is troublesome for the operator to search for the imaging protocol.

In the technique discussed in Japanese Patent Application Laid-Open No. 2011-50518, an image can be changed from the imaging protocol already used in imaging to an imaging protocol that has not yet been used in imaging. However, the technique discussed in Japanese Patent Application Laid-Open No. 2011-50518 is on the premise that the imaging protocol that has not yet been used is included in the examination order if the imaging protocol is changed. If the imaging protocol is desired to be changed when the imaging protocol that has not yet been used is not included in the examination order, the imaging protocol that has not yet been used needs to be added, an image is moved from the imaging protocol that has been already used in imaging to the imaging protocol that has not yet been used in imaging, and the imaging protocol that the image is moved needs to be deleted. It is sometimes troublesome for the operator to perform such a series of operations.

### SUMMARY OF THE INVENTION

According to an aspect of the present invention, a radiation imaging system capable of performing imaging based on an examination order including a plurality of imaging protocols includes an imaging unit configured to perform the imaging by a radiation generation unit irradiating an object with a radiation and a radiation detection unit detecting the radiation passing through the object based on the imaging protocols, an image processing unit configured to perform image processing on the captured image based on the imaging protocols used at the time of imaging by the imaging unit, a replacement source specification unit configured to specify a replacement source imaging protocol from the examination order based on an instruction from an operator, a replacement destination specification unit configured to specify one imaging protocol from among a plurality of replacement destination imaging protocols obtained based on information attached to the imaging protocol specified by the replacement source specification unit, and a replacement unit configured to replace the imaging protocol specified by the replacement source specification unit with the imaging protocol specified by the replacement destination specification unit.

Further features of the present invention will become apparent from the following description of exemplary embodiments (with reference to the attached drawings).

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 illustrates an example of an X-ray imaging system to which a radiation imaging system according to an exemplary embodiment of the present invention is applied.

FIG. 2 illustrates an example of a functional configuration of an X-ray imaging control unit according to a first exemplary embodiment.

FIG. 3 illustrates an example of an examination screen obtained before an imaging protocol is replaced according to the first and a second exemplary embodiment.

FIG. 4 illustrates an example of an examination screen obtained after an imaging protocol that is not yet used in imaging is replaced according to the first and second exemplary embodiments.

FIG. 5 illustrates an example of an examination screen obtained after the imaging protocol that is already used in imaging is replaced according to the first and second exemplary embodiments.

FIG. 6 illustrates an example of a screen for specifying a replacement destination of the imaging protocol that is not yet used in imaging according to the first exemplary embodiment.

FIG. 7 illustrates an example of a screen for specifying a replacement destination of the imaging protocol that is already used in imaging according to the first exemplary embodiment.

FIG. 8 is a flow chart illustrating an example of an imaging operation of the X-ray imaging apparatus according to the first exemplary embodiment of the present invention.

FIG. 9 is a flow chart illustrating an example of an operation for replacing an imaging protocol in the X-ray imaging apparatus according to the first exemplary embodiment.

FIG. 10 illustrates an example of a functional configuration of an X-ray imaging control unit according to the second exemplary embodiment.

FIG. 11 illustrates an example of a screen for specifying a replacement destination of the imaging protocol that is not yet used in imaging according to the second exemplary embodiment.

FIG. 12 illustrates an example of a screen for specifying a replacement destination of the imaging protocol that is already used in imaging according to the second exemplary embodiment.

FIG. 13 is a flow chart illustrating an example of an operation for replacing an imaging protocol in the X-ray imaging apparatus according to the second exemplary embodiment.

FIG. 14 illustrates an example of a functional configuration of an X-ray imaging control unit according to a third exemplary embodiment.

FIG. 15 illustrates an example of an examination screen obtained before an imaging protocol is replaced according to the third exemplary embodiment.

FIG. 16 illustrates an example of an examination screen obtained after the imaging protocol is replaced according to the third exemplary embodiment.

FIG. 17 is a flow chart illustrating an example of an operation for replacing an imaging protocol in the X-ray imaging apparatus according to the third exemplary embodiment.

FIG. 18 illustrates an example of a functional configuration of an X-ray imaging control unit according to a fourth exemplary embodiment.

FIG. 19 illustrates an example of an examination screen according to the fourth exemplary embodiment.

FIG. 20 illustrates an example of the examination screen according to the fourth exemplary embodiment.

FIG. 21 is a flow chart illustrating an example of an operation for replacing an imaging protocol in the X-ray imaging apparatus according to the fourth exemplary embodiment.

### DESCRIPTION OF THE EMBODIMENTS

Various exemplary embodiments, features, and aspects of the invention will be described in detail below with reference to the drawings.

An exemplary embodiment of a radiation imaging system and a processing method thereof according to the present invention is described below with reference to the accompanying drawings. In the exemplary embodiments described below, an example is cited where an X-ray is used as a radiation, the radiation is not limited to the X-ray, and an electromagnetic wave, α ray, β ray, or γ ray may be used.

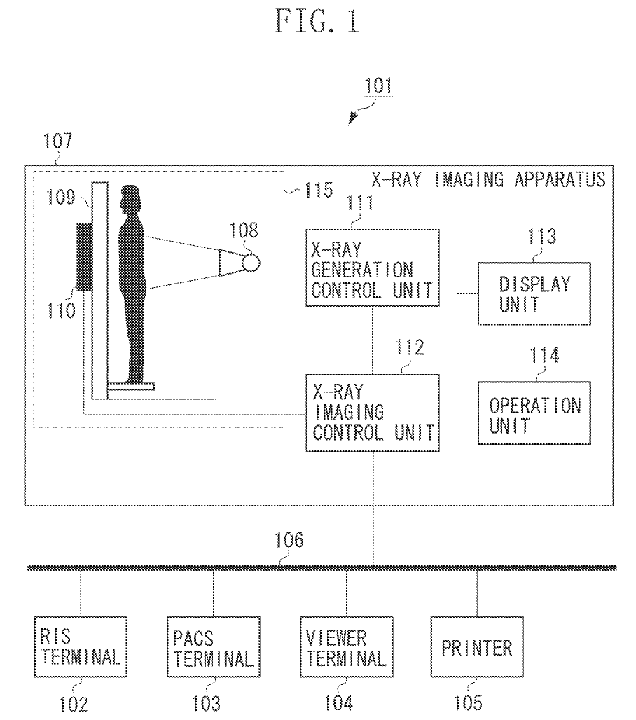

FIG. 1 illustrates an example of an X-ray imaging system 101 to which the radiation imaging system according to a first exemplary embodiment of the present invention is applied.

The X-ray imaging system 101 includes an RIS terminal 102, a picture archiving and communication system (PACS) terminal 103, a viewer terminal 104, a printer 105, and an X-ray imaging apparatus 107. These apparatuses are connected to one another via a communication unit 106 such as a network, for example.

The RIS terminal 102 is an information system in a radiology department. The X-ray imaging apparatus 107 captures an X-ray digital image (hereinafter referred to as a captured image). The PACS terminal 103 stores and manages the images captured by the X-ray imaging apparatus 107. The viewer terminal 104 and the printer 105 output (displays or prints) a diagnostic image.

The X-ray imaging apparatus 107 performs examination (imaging) based on an examination order including a plurality of the imaging protocols. Each of the imaging protocols defines imaging conditions and contents of image processing applying to the captured image. More specifically, the imaging protocol includes information about various types of parameters at the time of imaging and image processing and information about imaging execution, and is associated with imaging environment information (type of a sensor and an imaging orientation).

The X-ray imaging apparatus 107 includes an imaging unit 115, an X-ray generation control unit 111, an X-ray imaging control unit 112, a display unit 113, and an operation unit 114.

The imaging unit 115 is equipped with an X-ray tube 108, a sensor unit 109, and a sensor 110. The X-ray tube 108 functions as a radiation generation unit and irradiates an object (or, an examinee) with X-ray. The X-ray generation control unit 111 controls the generation of X-ray based on the imaging protocol. More specifically, a voltage is applied to the X-ray tube 108 based on the imaging conditions (parameters such as tube current, tube voltage, and irradiation time, for example) corresponding to the imaging protocol to generate the X-ray.

The sensor 110 functions as a radiation detection unit and detects the X-ray passing through the examinee. The sensor unit 109 performs analog-to-digital (A/D) conversion on an electric charge corresponding to an amount of the X-ray passing through the examinee detected by the sensor 110 and transfers the converted digital signal as a captured image to the X-ray imaging control unit 112. The X-ray imaging control unit 112 comprehensively controls X-ray imaging processing based on the imaging protocol. The X-ray imaging control unit 112 performs, for example, the image processing (correction processing, gradation processing, and frequency processing, for example) on the captured image. The image processing is performed using an image processing parameter corresponding to the imaging protocol at the time of imaging.

The display unit 113 includes a display, for example and displays a state of the system or the like for the operator. The display unit 113 displays an examination order received from the RIS terminal 102, for example, (or an examination order created by the operator). The operation unit 114 includes a keyboard, a mouse, and various buttons, for example, and inputs instructions from the operator to the apparatus. The operation unit 114 inputs the specification of the imaging protocol in a replacement source and a replacement destination, for example, to the apparatus.

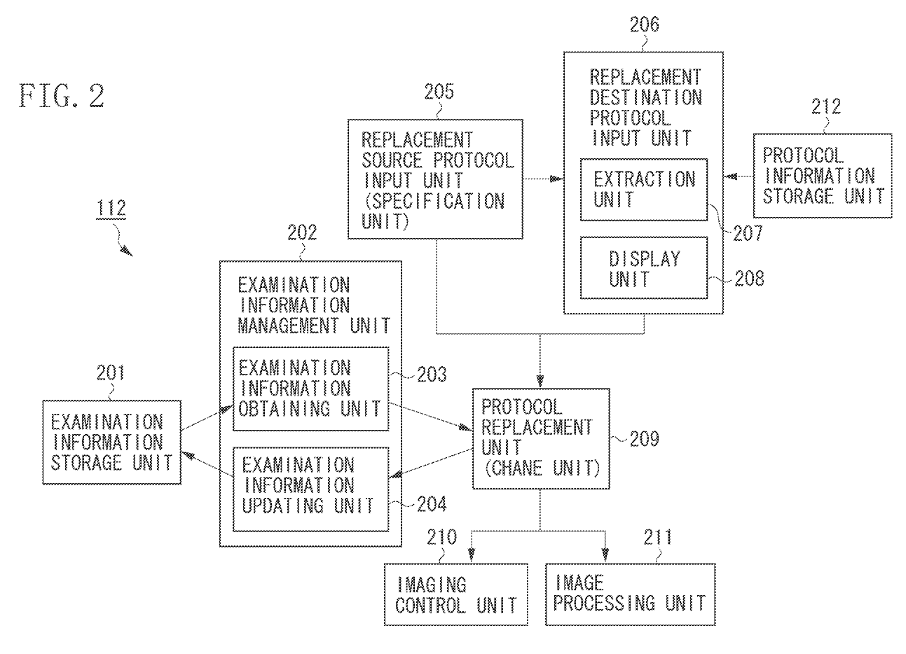

An example of a functional configuration for realizing the first exemplary embodiment in the X-ray imaging control unit 112 illustrated in FIG. 1 is described below with reference to FIG. 2.

The X-ray imaging control unit 112 includes an examination information management unit 202, an examination information storage unit 201, a replacement source protocol input unit 205, a replacement destination protocol input unit 206, a protocol replacement unit 209, an imaging control unit 210, and an image processing unit 211.

The image processing unit 211 performs the image processing on the captured image. As described above, the image processing is performed using the image processing parameter corresponding to the imaging protocol.

The replacement source protocol input unit 205 specifies the imaging protocol to be replaced (replacement source) from among the imaging protocols included in the examination order which is currently executed. The specification of the imaging protocol by the replacement source protocol input unit 205 is performed based on an instruction from the operator via the operation unit 114.

The replacement destination protocol input unit 206 specifies the imaging protocol to be replaced (replacement destination) from among the imaging protocols stored in a protocol information storage unit 212. The replacement destination protocol input unit 206 includes an extraction unit 207 and a display unit 208. When the replacement destination protocol input unit 206 receives information about the replacement source imaging protocol from the replacement source protocol input unit 205, if the replacement source imaging protocol is not yet used in imaging, the extraction unit 207 extracts all imaging protocols stored in the protocol information storage unit 212 as candidates to be replaced. Whereas if the replacement source imaging protocol is already used in imaging, the extraction unit 207 extracts only the imaging protocol including the same imaging environment information (type of a sensor and an imaging orientation) as the replacement source imaging protocol from the imaging protocols stored in the protocol information storage unit 212 as candidates to be replaced. The display unit 208 displays the imaging protocols as the candidates to be replaced which are extracted by the extraction unit 207. The display unit 208 preferentially displays the imaging protocol being a region near to the imaging region of the replacement source imaging protocol. The display of the display unit 208 is performed via the display unit 113. The specification of the replacement destination protocol input unit 206 is performed based on an instruction from the operator via the operation unit 114.

The examination information management unit 202 manages various pieces of information concerning examination which is currently executed based on the examination order obtained from the RIS terminal 102 (or manually produced by the operator). The examination information management unit 202 includes an examination information obtaining unit 203 and an examination information updating unit 204. The examination information obtaining unit 203 obtains information concerning examination which is currently executed from the examination information storage unit 201 and the examination information updating unit 204 updates the information concerning examination stored in the examination information storage unit 201.

The protocol replacement unit 209 replaces the replacement source imaging protocol specified by the replacement source protocol input unit 205 with the replacement destination imaging protocol specified by the replacement destination protocol input unit 206. If the replacement source imaging protocol is already used in imaging, the captured image of the replacement source imaging protocol is associated with the replacement destination imaging protocol. Accordingly, the image processing unit 211 performs the image processing on the captured image using the image processing parameter corresponding to the replacement destination imaging protocol.

The imaging control unit 210 controls an imaging operation based on the imaging protocol. For example, if the imaging protocol is replaced by the protocol replacement unit 209, the imaging control unit 210 controls the execution of the imaging operation based on the replaced imaging protocol.

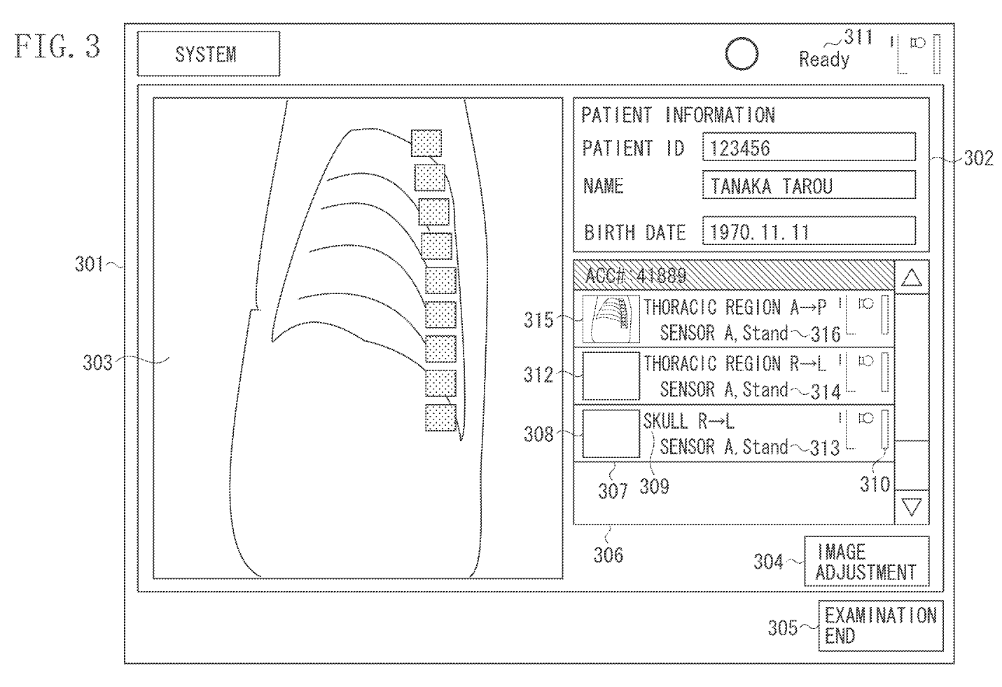

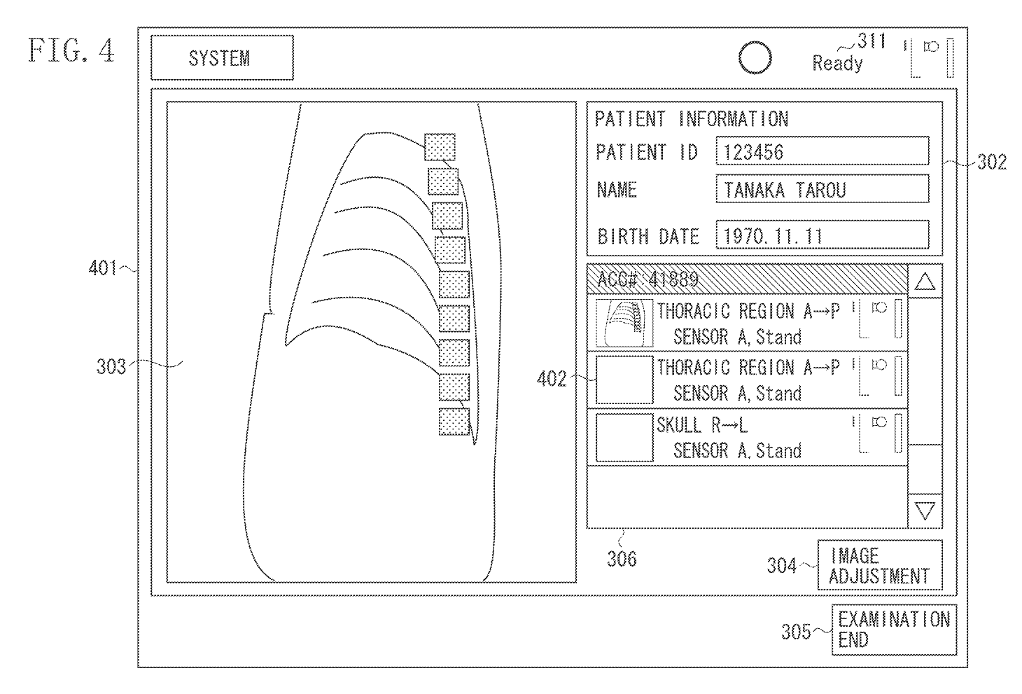

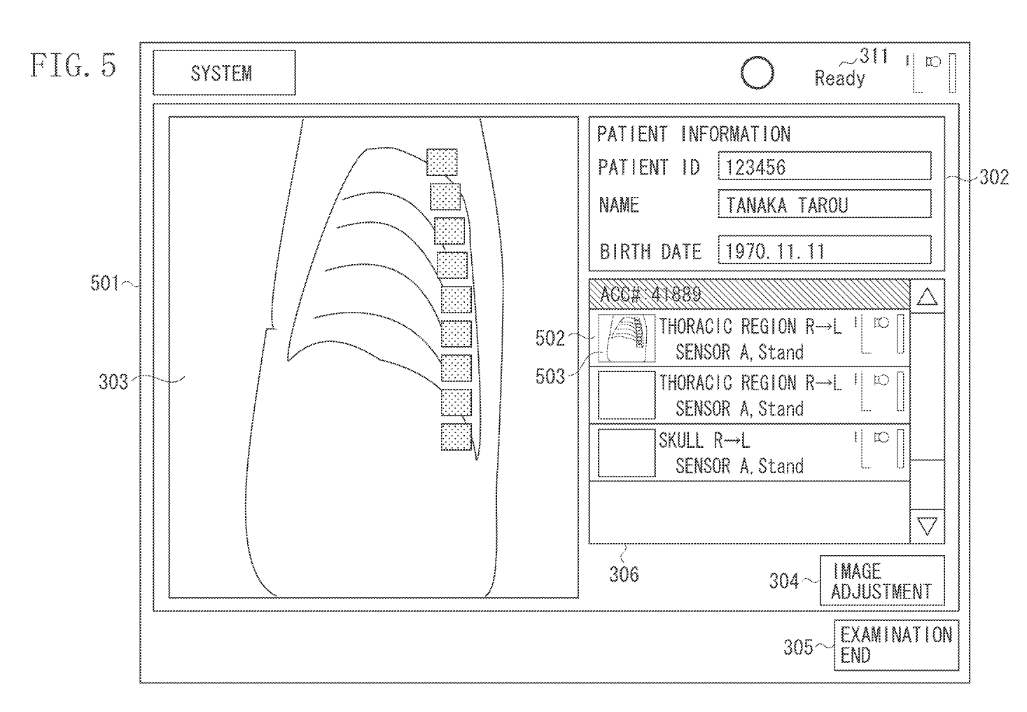

FIGS. 3, 4, and 5 illustrate examples of imaging screens. FIG. 3 illustrates a screen 301 obtained before the imaging protocol is replaced. FIG. 4 illustrates a screen 401 obtained after the imaging protocol that is not yet used in imaging is replaced. FIG. 5 illustrates a screen 501 obtained after the imaging protocol that is already used in imaging is replaced.

In FIGS. 3, 4, and 5, the imaging screen includes an examinee information display area 302 for displaying information about an examinee, a preview image display area 303 for displaying a captured image, an image adjustment button 304 for instructing the adjustment of an image processing parameter of the captured image which is currently previewed, an examination end button 305 for instructing the end of examination, and an examination order display area 306 for indicating information about the examination order which is currently executed. The examination order display area 306 is provided with a plurality of imaging protocol buttons 307.

The imaging protocol buttons 307 include a thumbnail display area 308 for displaying the thumbnail of the captured image, a name display area 309 for displaying the name of the imaging region, a sensor-type display area 310 for displaying information about the type of a sensor such as a standing position or a lying position, and an imaging environment information display area 313 for displaying information about the type of the sensor and an imaging orientation. The imaging protocol buttons 307 also indicates states where the imaging is already ended (during the preview), in preparation, and can be performed.

The imaging screens illustrated in FIGS. 3, 4, and 5 are also provided with a sensor-state display area 311 for indicating the state of the sensor regarding the imaging protocol that is currently executed. In the imaging screens illustrated in FIGS. 3, 4, and 5, the states are indicated in characters, but the states may be indicated by a graphic user interface (GUI) such that the color of the button is changed or animation is displayed.

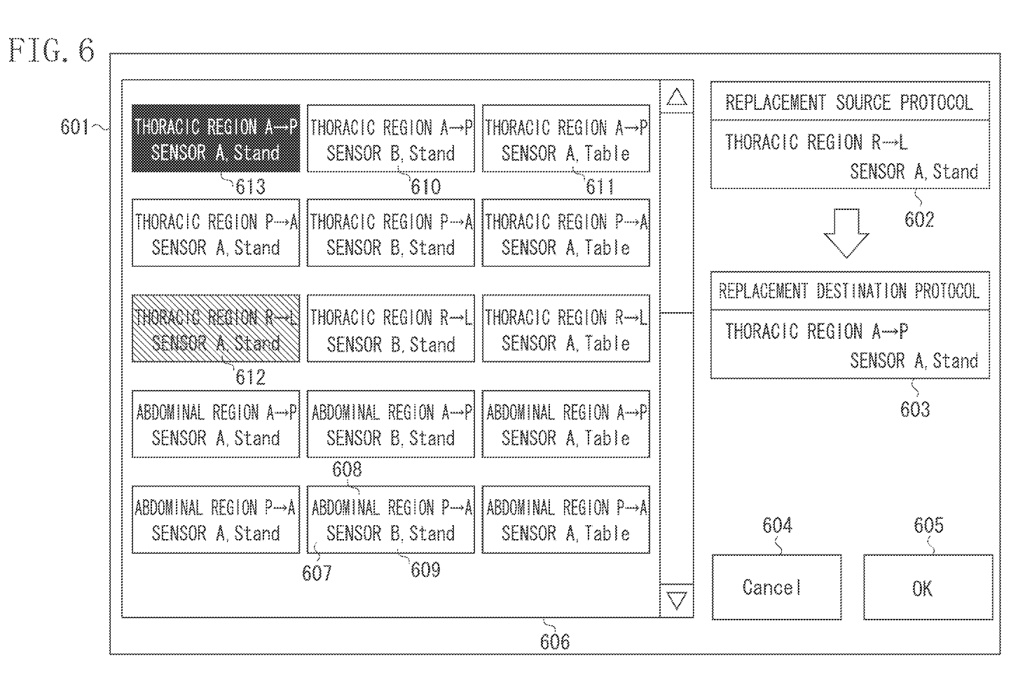

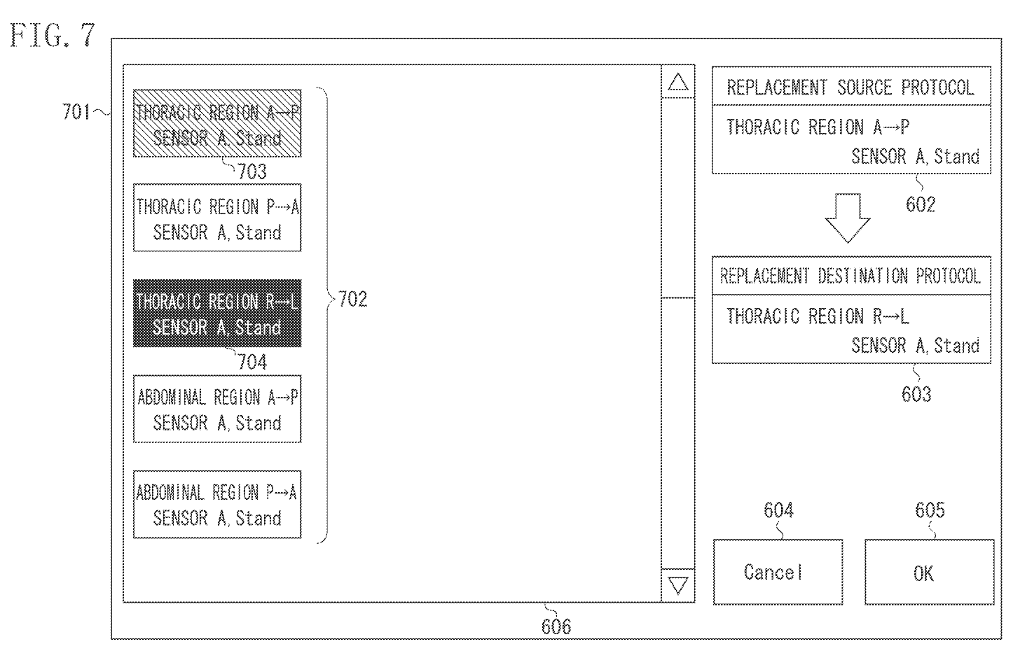

FIGS. 6 and 7 illustrate examples of a replacement destination imaging protocol specifying screen. FIG. 6 illustrates a replacement destination imaging protocol specifying screen 601 in a case where the imaging protocol that is not yet used in imaging is replaced. FIG. 7 illustrates a replacement destination imaging protocol specifying screen 701 in a case where the imaging protocol that is already used in imaging is replaced.

The screen illustrated in FIGS. 6 and 7 include a replacement source imaging protocol display area 602, a replacement destination imaging protocol display area 603, a cancel button 604 for cancelling the replacement of the imaging protocol, a determination button 605 for determining the replacement of the imaging protocol, and a replacement destination imaging protocol candidate display area 606. The replacement destination imaging protocol candidate display area 606 includes a plurality of imaging protocol buttons 607 in FIG. 6.

The imaging protocol buttons 607 are provided with a name display area 608 for displaying the name of the imaging region and an imaging environment information display area 609.

A specific example of a processing flow according to the first exemplary embodiment is described below.

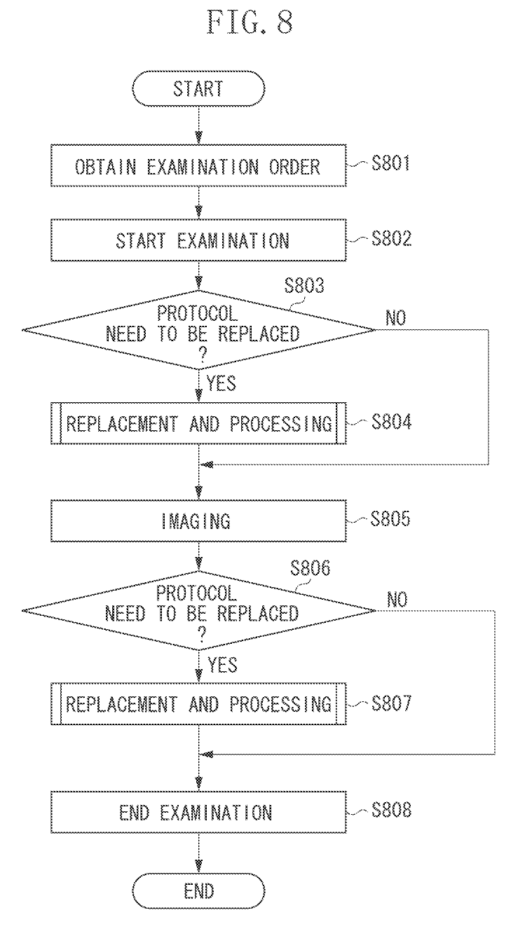

A series of flow from the start to the end of an examination is described below with reference to FIG. 8. In step S801, the examination information management unit 202 obtains the examination order obtained from the RIS terminal 102 (or manually generated by an operator). In step S802, the examination is started, and the examination screen 301 or 501 is displayed on the display unit 113. In step S803, if the imaging protocol needs to be replaced, that is, for example, if the operator (i.e., a radiological technician) finds that an erroneous imaging protocol is included in the examination order before imaging (YES in step S803), the processing proceeds to step S804 to perform the replacement and process of the imaging protocol.

If the imaging protocol does not need to be replaced (NO in step S803), the processing proceeds to step S805. In step S805, the operator presses an irradiation button. Then, in the X-ray imaging apparatus 107, the X-ray tube 108 irradiates the object with X-ray, and the sensor 110 detects the X-ray passing through the object. A captured image resultantly obtained is subjected to the image processing such as correction processing, gradation processing, and frequency processing by the X-ray imaging control unit 112.

In step S806, when the captured image which is subjected to the image processing is displayed on the display unit 113, the operator confirms the image. As a result of the confirmation, if the imaging protocol needs to be replaced, that is, for example, if the operator finds that the imaging protocol is in error (YES in step S806), the processing proceeds to step S807 to perform the replacement and process of the imaging protocol. If the imaging protocol does not need to be replaced (NO in step S806), the processing proceeds to step S808. In step S808, the operator presses the examination end button 305 to end the examination.

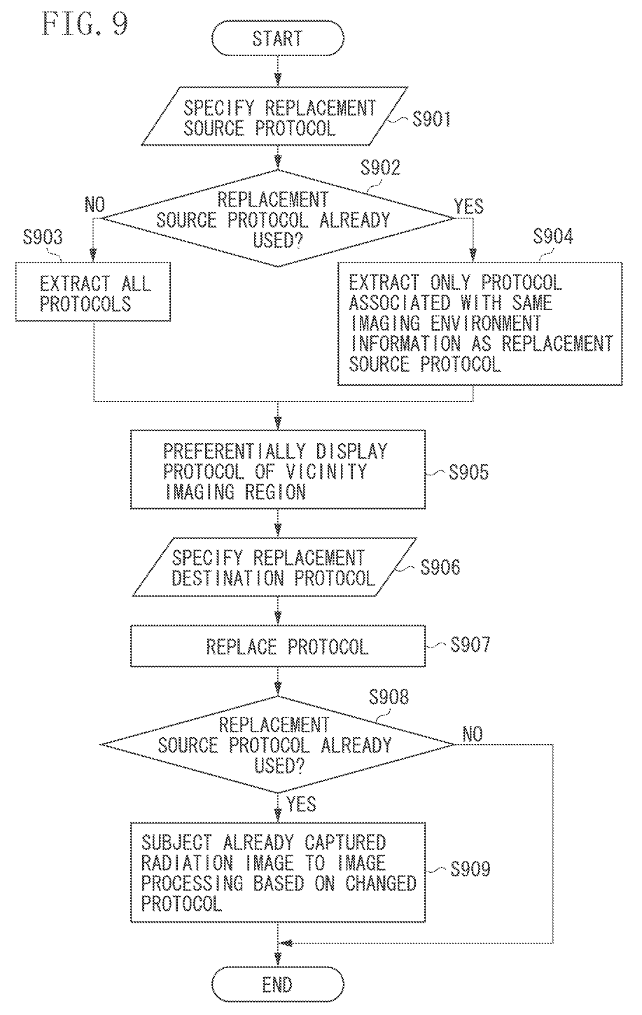

A processing flow for replacing the imaging protocol that is not yet used in imaging in step S804 or S807 is described below with reference to FIG. 9. Assuming that the imaging protocol that is not yet used in imaging and is included in the examination order needs to be replaced. For example, in step S803 (S806), assuming that the operator finds that “Thoracic region R→L” is mistaken for “Thoracic region A→P” in the imaging region of the imaging protocol 312 that is not yet used in imaging. The operator determines that the imaging protocol needs to be replaced, and the processing proceeds to step S804 (S807) perform the replacement operation of the imaging protocol. A flow of specific processing in step S804 (S807) is described in FIG. 9.

In step S901, the operator specifies the imaging protocol 312 that is not yet used in imaging as the replacement source imaging protocol via an operation of the operation unit 114. Then, information about the imaging protocol 312 that is not yet used in imaging is input to the replacement source protocol input unit 205. In step S902, the extraction unit 207 receives the information input to the replacement source protocol input unit 205 and determines whether the replacement source imaging protocol is not yet used or already used in imaging. Since the imaging protocol 312 input to the replacement source protocol input unit 205 is not yet used (NO in step S902), the processing proceeds to step S903. In step S903, the extraction unit 207 extracts all the imaging protocols stored in the protocol information storage unit 212 as candidates for replacement destination imaging protocols. Then, the processing proceeds to step S905.

In step S905, the display unit 208 displays the imaging protocols extracted by the extraction unit 207 via the display unit 113. More specifically, the display unit 208 displays the replacement destination imaging protocol specifying screen 601 for the imaging protocol that is not yet used in imaging on the display unit 113 and displays the imaging protocols extracted by the extraction unit 207 in the replacement destination imaging protocol candidate display area 606. The imaging region of the replacement source imaging protocol is a thoracic region, so that the display unit 208 performs control to preferentially display the imaging protocol associated with the imaging region in the vicinity of the thoracic region in the replacement destination imaging protocol candidate display area 606. As illustrated in FIG. 6 as an example, the imaging protocols of which imaging region are a thoracic region are displayed above the replacement destination imaging protocol candidate display area 606 so that the operator can easily select an imaging protocol.

The replacement source imaging protocol input in step S901 is the imaging protocol 312 that is not yet used in imaging and the imaging environment information thereof is “Sensor A, Stand” as illustrated in an imaging environment information display area 314. Whereas, in FIG. 6, an imaging protocol button 610 indicates that an imaging environment information is “Sensor B, Stand”, and an imaging protocol button 611 indicates that an imaging environment information is “Sensor A, Table”. This shows that the candidates for the replacement destination imaging protocols extracted by the extraction unit 207 in step S903 are all the imaging protocols stored in the protocol information storage unit 212 irrespective of the imaging environment information of “Sensor A, Stand” of the replacement source imaging protocol 312. The display unit 208 displays information about the replacement source imaging protocol 312 input in step S901 in the replacement source imaging protocol display area 602 and highlights an imaging protocol button 612 similar to the replacement source imaging protocol. Then, the processing proceeds to step S906.

In step S906, the operator specifies a desired replacement destination imaging protocol 613 as a replacement destination imaging protocol via an operation of the operation unit 114. The display unit 208 displays information about the specified replacement destination imaging protocol 613 in the replacement destination imaging protocol display area 603 and highlights the replacement destination imaging protocol 613. Then, the processing proceeds to step S907. In step S907, if the replacement of the imaging protocol is cancelled, the operator presses the cancel button 604 via an operation of the operation unit 114 to cancel the replacement of the imaging protocol. Accordingly, the screen returns to the examination screen 301.

If the replacement of the imaging protocol is determined, the operator presses the determination button 605 via an operation of the operation unit 114. By operating the determination button 605, the replacement destination imaging protocol 613 is input to the replacement destination protocol input unit 206. At this point, the protocol replacement unit 209 receives information about the replacement source and replacement destination imaging protocols from the replacement source protocol input unit 205 and the replacement destination protocol input unit 206. The protocol replacement unit 209 obtains examination information from the examination information storage unit 201 via the examination information obtaining unit 203. The protocol replacement unit 209 replaces the replacement source imaging protocol with the replacement destination imaging protocol in the imaging protocol included in the obtained examination information.

Then, the protocol replacement unit 209 updates the examination information stored in the examination information storage unit 201 via the examination information updating unit 204. If the replacement source imaging protocol is ready to be used in imaging, the protocol replacement unit 209 delivers the information about the replacement destination imaging protocol to the imaging control unit 210. The imaging control unit 210 performs imaging preparation control based on the received replacement destination imaging protocol. Then, the display unit 113 switches the display to the examination screen 401. As illustrated in an imaging protocol 402 in FIG. 4, the replacement source imaging protocol 312 is replaced with the replacement destination imaging protocol 613 specified in step S906. As described above, according to the first exemplary embodiment, the imaging protocol that is not yet used in imaging can be replaced.

A processing flow for replacing the imaging protocol that is already used in imaging in step S804 or S807 is described below with reference to FIG. 9. Assuming that the imaging protocol that is already used in imaging and is included in the examination order needs to be replaced. For example, in step S803 (S806), assuming that the operator finds that “Thoracic region A→P” is mistaken for “Thoracic region R→L” in the imaging region of the imaging protocol 315 that is already used in imaging. The operator determines that the imaging protocol needs to be replaced, and the processing proceeds to step S804 (S807) perform the replacement operation of the imaging protocol. A flow of specific processing in step S804 (S807) is described in FIG. 9.

In step S901, the operator specifies the imaging protocol 315 that is already used in imaging as the replacement source imaging protocol via an operation of the operation unit 114. Then, information about the imaging protocol 315 that is already used in imaging is input to the replacement source protocol input unit 205. In step S902, the extraction unit 207 receives the information input to the replacement source protocol input unit 205 and determines whether the replacement source imaging protocol is not yet used or already used in imaging. Since the imaging protocol 315 input to the replacement source protocol input unit 205 is already used (YES in step S902), the processing proceeds to step S904. In step S904, the extraction unit 207 extracts only the imaging protocol including the same imaging environment information as the replacement source imaging protocol from the imaging protocols stored in the protocol information storage unit 212 as a candidate for the replacement destination imaging protocol. More specifically, the imaging protocol 315 is specified as the replacement source imaging protocol in step S901. The imaging environment information of the imaging protocol 315 is “Sensor A, Stand” as indicated in an imaging environment information display area 316, so that the extraction unit 207 extracts only the imaging protocol of which imaging environment information is “Sensor A, Stand” among the image protocols stored in the protocol information storage unit 212. Then, the processing proceeds to step S905.

In step S905, the display unit 208 displays the imaging protocol extracted by the extraction unit 207 via the display unit 113. More specifically, the display unit 208 displays the replacement destination imaging protocol specifying screen 701 for the imaging protocol that is already used in imaging on the display unit 113 and displays the imaging protocol extracted by the extraction unit 207 in the replacement destination imaging protocol candidate display area 606. The imaging region of the replacement source imaging protocol is a thoracic region, so that the display unit 208 performs control to preferentially display the imaging protocol associated with the imaging region in the vicinity of the thoracic region in the replacement destination imaging protocol candidate display area 606. As illustrated in FIG. 7 as an example, the imaging protocols of which imaging region are a thoracic region are displayed above the replacement destination imaging protocol candidate display area 606 so that the operator can easily select an imaging protocol.

The replacement source imaging protocol input in step S901 is the imaging protocol 315 that is already used in imaging and the imaging environment information thereof is “Sensor A, Stand” as illustrated in the imaging environment information display area 316. As illustrated in imaging protocols 702, the imaging environment information of all of the imaging protocols displayed in the replacement destination imaging protocol candidate display area 606 is “Sensor A, Stand.” This means that the extraction unit 207 extracts only the imaging protocol including the same imaging environment information of “Sensor A, Stand” as the replacement source imaging protocol 315 as a candidate for the replacement destination imaging protocol in step S904. The display unit 208 displays information about the replacement source imaging protocol 315 input in step S901 in the replacement source imaging protocol display area 602 and highlights an imaging protocol button 703 similar to the replacement source imaging protocol. Then, the processing proceeds to step S906.

In step S906, the operator specifies a desired replacement destination imaging protocol 704 as a replacement destination imaging protocol via an operation of the operation unit 114. The display unit 208 displays information about the specified replacement destination imaging protocol 704 in the replacement destination imaging protocol display area 603 and highlights the replacement destination imaging protocol 704. Then, the processing proceeds to step S907. In step S907, if the replacement of the imaging protocol is cancelled, the operator presses the cancel button 604 via an operation of the operation unit 114 to cancel the replacement of the imaging protocol. Accordingly, the screen returns to the examination screen 301.

If the replacement of the imaging protocol is determined, the operator presses the determination button 605 via an operation of the operation unit 114. By operating the determination button 605, the replacement destination imaging protocol 704 is input to the replacement destination protocol input unit 206. At this point, the protocol replacement unit 209 receives information about the replacement source and replacement destination imaging protocols from the replacement source protocol input unit 205 and the replacement destination protocol input unit 206. The protocol replacement unit 209 obtains examination information from the examination information storage unit 201 via the examination information obtaining unit 203. The protocol replacement unit 209 replaces the replacement source imaging protocol with the replacement destination imaging protocol in the imaging protocol included in the obtained examination information. At the same time, the captured image of the replacement source imaging protocol is associated with the replacement destination imaging protocol.

Then, the protocol replacement unit 209 updates the examination information stored in the examination information storage unit 201 via the examination information updating unit 204. The protocol replacement unit 209 delivers the information about the replacement destination imaging protocol to the image processing unit 211. The image processing unit 211 performs the image processing again on the image captured by the replacement source imaging protocol (or the image not yet subjected to the image processing) using the image processing parameter associated with the replacement destination imaging protocol. The display unit 113 switches the display to the examination screen 501. As illustrated in an imaging protocol 502 in FIG. 5, the replacement source imaging protocol 315 is replaced with the replacement destination imaging protocol 704 specified in step S906. In addition, the captured image subjected again to the image processing by the image processing unit 211 is displayed in the preview image display area 303 and a thumbnail display area 503. As described above, according to the first exemplary embodiment, the imaging protocol that is already used in imaging can be replaced.

According to the first exemplary embodiment, an erroneous imaging protocol can be easily replaced with another imaging protocol at the time of executing imaging based on the examination order. Since an imaging protocol near to an imaging region is preferentially displayed, the operator who wants to perform replacement to the imaging protocol near to the imaging region can easily specify the replacement destination. In addition, if the operator wants to replace the imaging protocol that is already used in imaging, a replaceable imaging protocol needs to include the same imaging environment information as the replacement source imaging protocol, however, only the imaging protocol with the same imaging environment information is displayed as a candidate for the replacement destination imaging protocol and can be specified, which does not burden the operator with a heavy load.

A second exemplary embodiment is described below.

An X-ray imaging system to which the radiation imaging system according to the second exemplary embodiment is applied is similar to the one according to the first exemplary embodiment illustrated in FIG. 1.

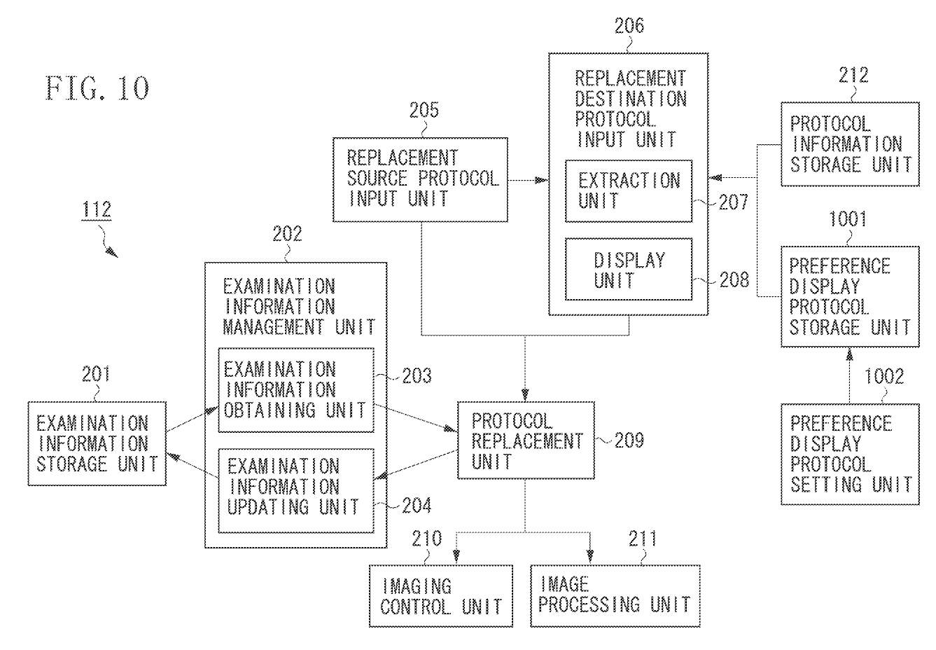

An example of a functional configuration for realizing the second exemplary embodiment in the X-ray imaging control unit 112 illustrated in FIG. 1 is described below with reference to FIG. 10. In FIG. 10, the components similar to those in FIG. 2 which describes the first exemplary embodiment are given the same reference numerals. A preference display protocol storage unit 1001 stores information about an imaging protocol which is preferentially displayed when candidates for a replacement destination imaging protocol is displayed on the display unit 208. A preference display protocol setting unit 1002 sets a preferentially displayed protocol set by the operator via an operation of the operation unit 114 and reflects the setting in the preference display protocol storage unit 1001.

Imaging screens according to the second exemplary embodiment are similar to those in FIGS. 3, 4, and 5 according to the first exemplary embodiment.

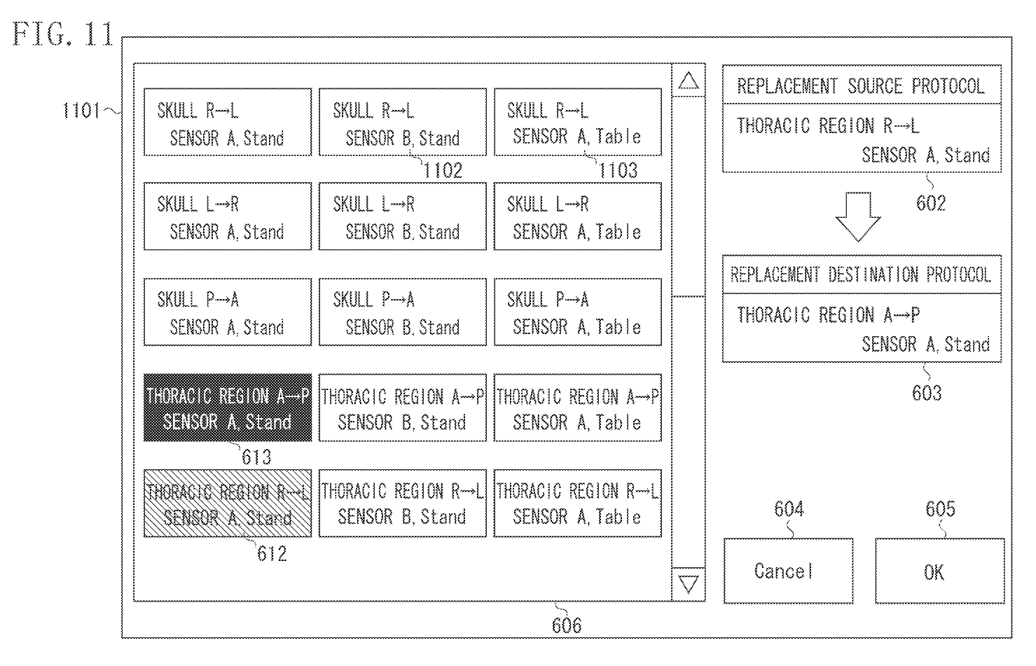

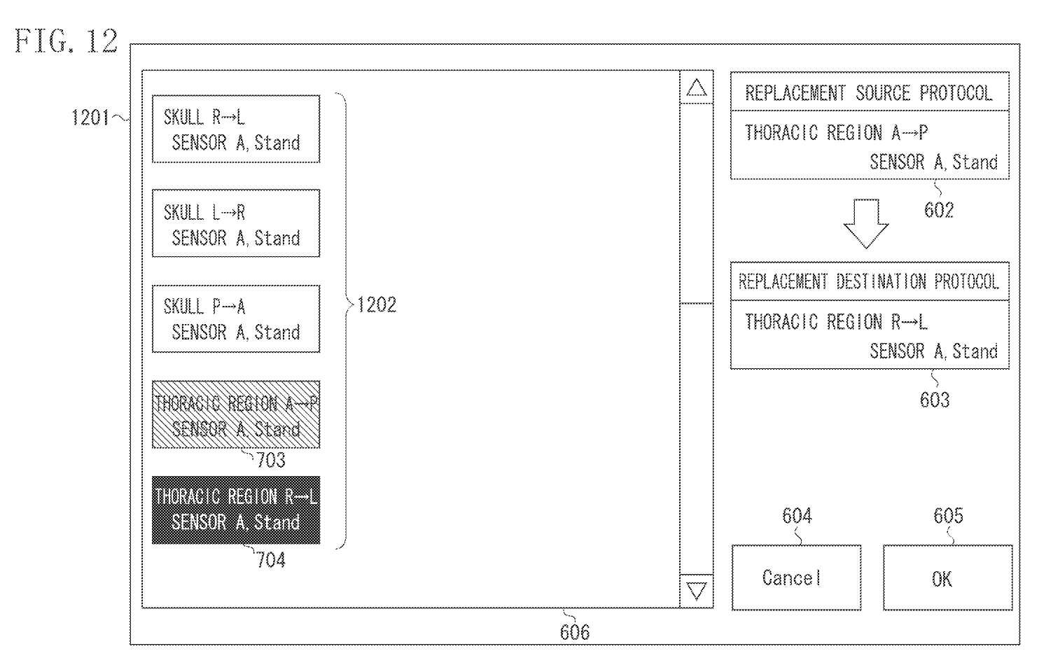

FIGS. 11 and 12 illustrate examples of a replacement destination imaging protocol specifying screen according to the second exemplary embodiment. FIG. 11 illustrates a replacement destination imaging protocol specifying screen 1101 in a case where the imaging protocol that is not yet used in imaging is replaced. FIG. 12 illustrates a replacement destination imaging protocol specifying screen 1201 in a case where the imaging protocol that is already used in imaging is replaced. In FIGS. 11 and 12, the components similar to those in FIGS. 6 and 7 which describe the first exemplary embodiment are given the same reference numerals.

A specific example of a processing flow according to the second exemplary embodiment is described below.

A series of flow from the start to the end of an examination according to the second exemplary embodiment is similar to the one according to the first exemplary embodiment as illustrated in FIG. 8.

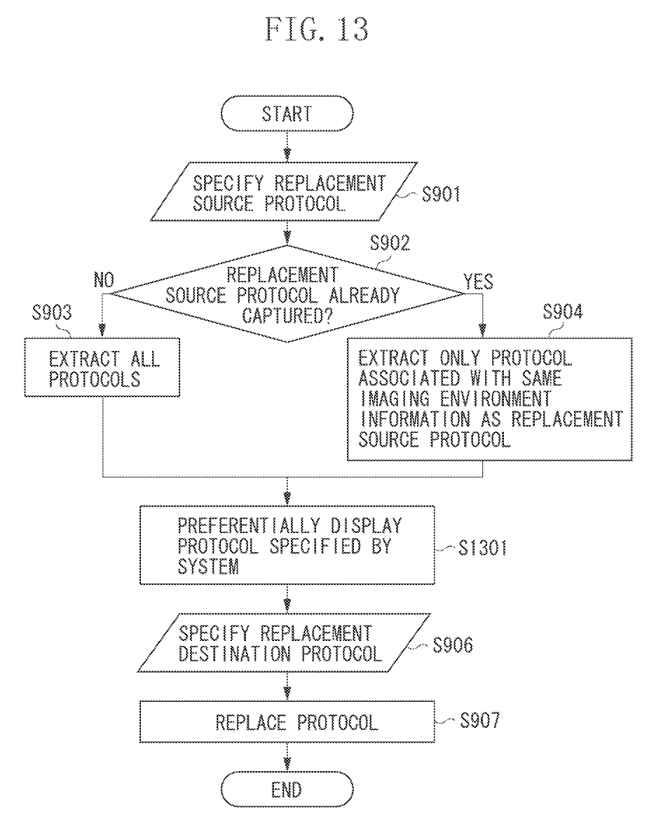

A processing flow for replacing the imaging protocol that is not yet used in imaging in step S804 or S807 in FIG. 8 is described below with reference to FIG. 13. According to the second exemplary embodiment, the processing performed in step S804 or S807 is illustrated in FIG. 13. In FIG. 13, the configuration similar to the one in FIG. 9 describing the first exemplary embodiment is given the same reference numeral. The processing flow for replacing the imaging protocol that is not yet used in imaging is also similar to the one in FIG. 9 describing the first exemplary embodiment except for the processing in step S905.

In step S1301, the display unit 208 displays the imaging protocol extracted by the extraction unit 207 via the display unit 113. More specifically, the display unit 208 displays a replacement destination imaging protocol specifying screen 1101 for the imaging protocol that is not yet used in imaging on the display unit 113 and displays the imaging protocols extracted by the extraction unit 207 in the replacement destination imaging protocol candidate display area 606. At this point, assuming that an operator sets the imaging protocol of which imaging region is a skull as a preferentially displayed imaging protocol via an operation of the operation unit 114 by the preference display protocol setting unit 1002 before the start of examination, for example, and the setting contents are stored in the preference display protocol storage unit 1001. The display unit 208 performs control to preferentially display a preference display imaging protocol stored in the preference display protocol storage unit 1001. As illustrated in FIG. 11 as an example, the imaging protocols of which imaging region are a skull set by the preference display protocol setting unit 1002 are displayed above the replacement destination imaging protocol candidate display area 606 so that the operator can easily select an imaging protocol.

The replacement source imaging protocol input in step S901 is the imaging protocol 312 that is not yet used in imaging and the imaging environment information thereof is “Sensor A, Stand” as illustrated in an imaging environment information display area 314. Whereas, in FIG. 11, an imaging protocol button 1102 indicates that imaging environment information is “Sensor B, Stand”, and an imaging protocol button 1103 indicates that imaging environment information is “Sensor A, Table”. This shows that the candidates for the replacement destination imaging protocols extracted by the extraction unit 207 in step S903 are all the imaging protocols stored in the protocol information storage unit 212 irrespective of the imaging environment information of “Sensor A, Stand” of the replacement source imaging protocol 312. The processing flow for replacing the imaging protocol that is not yet used in imaging is also similar to the one in FIG. 9 describing the first exemplary embodiment except for the replacement of the processing in step S905 with step S1301, so that the imaging protocol that is not yet used in imaging can be replaced according to the second exemplary embodiment.

A processing flow for replacing the imaging protocol that is already used in imaging in step S804 or S807 in FIG. 8 is described below with reference to FIG. 13. According to the second exemplary embodiment, the processing performed in step S804 or S807 is illustrated in FIG. 13. In FIG. 13, the configuration similar to the one in FIG. 9 describing the first exemplary embodiment is given the same reference numeral. The processing flow for replacing the imaging protocol that is already used in imaging is also similar to the one in FIG. 9 describing the first exemplary embodiment except for the processing in step S905.

In step S1301, the display unit 208 displays the imaging protocol extracted by the extraction unit 207 via the display unit 113. More specifically, the display unit 208 displays the replacement destination imaging protocol specifying screen 1201 for the imaging protocol that is already used in imaging on the display unit 113 and displays the imaging protocol extracted by the extraction unit 207 in the replacement destination imaging protocol candidate display area 606. At this point, assuming that the operator sets the imaging protocol of which imaging region is a skull as a preferentially displayed imaging protocol via an operation of the operation unit 114 by the preference display protocol setting unit 1002 before the start of examination, for example, and the setting contents are stored in the preference display protocol storage unit 1001. The display unit 208 performs control to preferentially display a preference display imaging protocol stored in the preference display protocol storage unit 1001. As illustrated in FIG. 12 as an example, the imaging protocols of which imaging region are a skull set by the preference display protocol setting unit 1002 are displayed above the replacement destination imaging protocol candidate display area 606 so that the operator can easily select an imaging protocol.

The replacement source imaging protocol input in step S901 is the imaging protocol 315 that is already used in imaging and the imaging environment information thereof is “Sensor A, Stand” as illustrated in the imaging environment information display area 316. As illustrated in imaging protocols 1202, the imaging environment information of all of the imaging protocols displayed in the replacement destination imaging protocol candidate display area 606 is “Sensor A, Stand.” This means that the extraction unit 207 extracts only the imaging protocol including the same imaging environment information of “Sensor A, Stand” as the replacement source imaging protocol 315 as a candidate for the replacement destination imaging protocol in step S904. The processing flow for replacing the imaging protocol that is already used in imaging is also similar to the one in FIG. 9 describing the first exemplary embodiment except for the replacement of the processing in step S905 with step S1301, so that the imaging protocol that is already used in imaging can be replaced according to the second exemplary embodiment.

According to the second exemplary embodiment, an erroneous imaging protocol can be easily replaced with another imaging protocol at the time of executing imaging based on the examination order. Since an imaging protocol set to be preferentially displayed is preferentially displayed, the operator who wants to specify a replacement destination from among the specific imaging protocols can easily specify the replacement destination. In addition, if the operator wants to replace the imaging protocol that is already used in imaging, a replaceable imaging protocol needs to include the same imaging environment information as the replacement source imaging protocol, however, only the imaging protocol with the same imaging environment information is displayed as a candidate for the replacement destination imaging protocol and can be specified, which does not burden the operator with a heavy load.

A third exemplary embodiment is described below.

An X-ray imaging system to which the radiation imaging system according to the third exemplary embodiment is applied is similar to the one according to the first exemplary embodiment illustrated in FIG. 1.

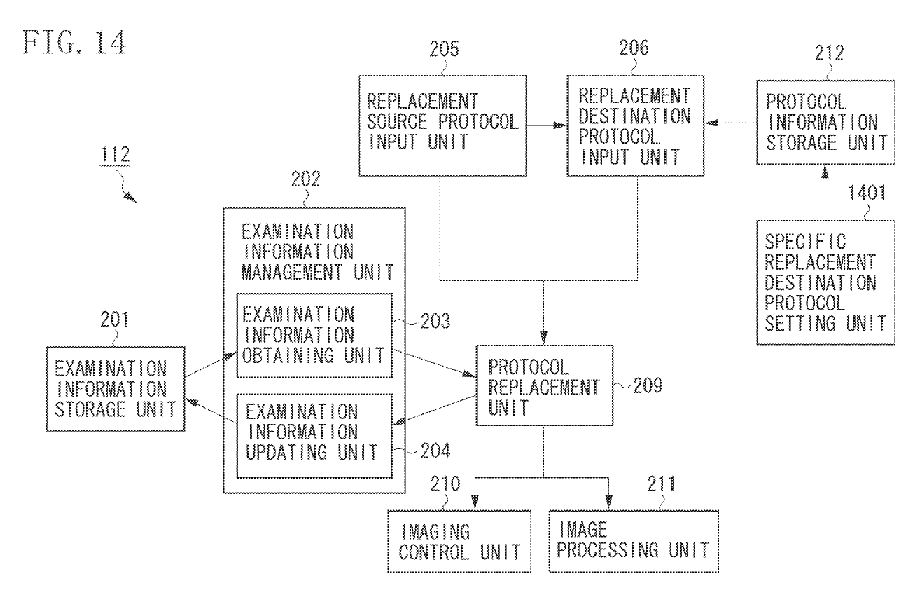

An example of a functional configuration for realizing the third exemplary embodiment in the X-ray imaging control unit 112 illustrated in FIG. 1 is described below with reference to FIG. 14. In FIG. 14, the components similar to those in FIG. 2 which describes the first exemplary embodiment are given the same reference numerals. Each of imaging protocols stored in the protocol information storage unit 212 is associated with a specific replacement destination imaging protocol. The specific replacement destination imaging protocol of each of the imaging protocols is set in a specific replacement destination protocol setting unit 1401 by the operator via an operation of the operation unit 114, and the setting is reflected in the protocol information storage unit 212. However, the captured image is different in an image size and the image processing method depending on the type of a sensor, so that the specific replacement destination imaging protocol which can be set by the specific replacement destination protocol setting unit 1401 is limited to the imaging protocol associated with the same imaging environment information as the replacement destination imaging protocol.

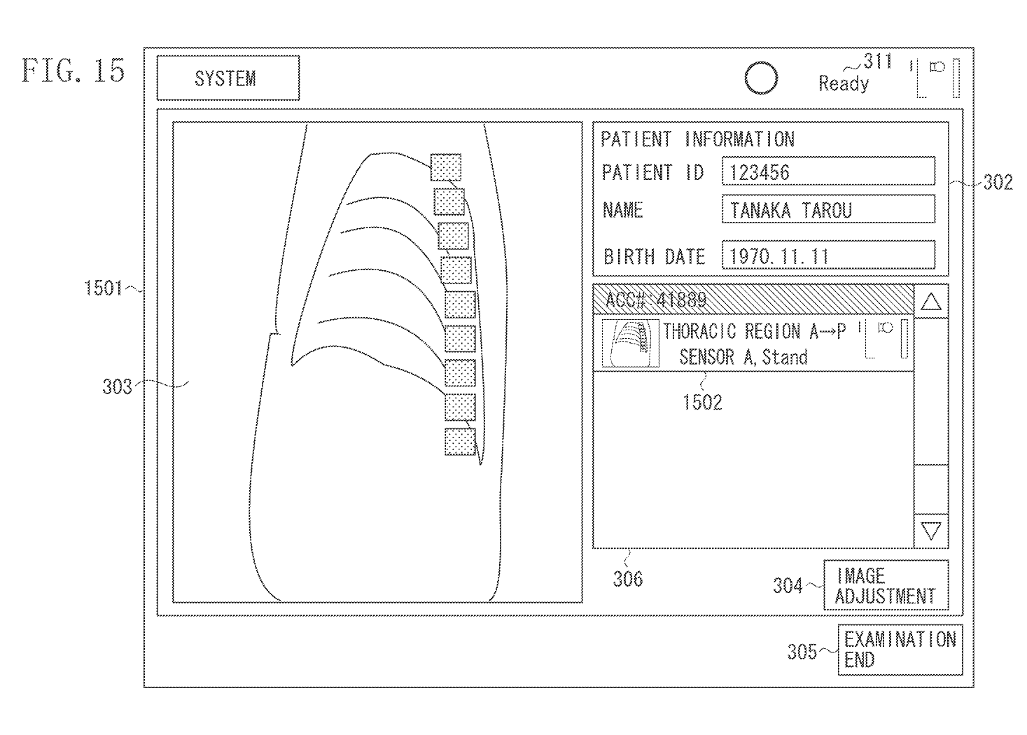

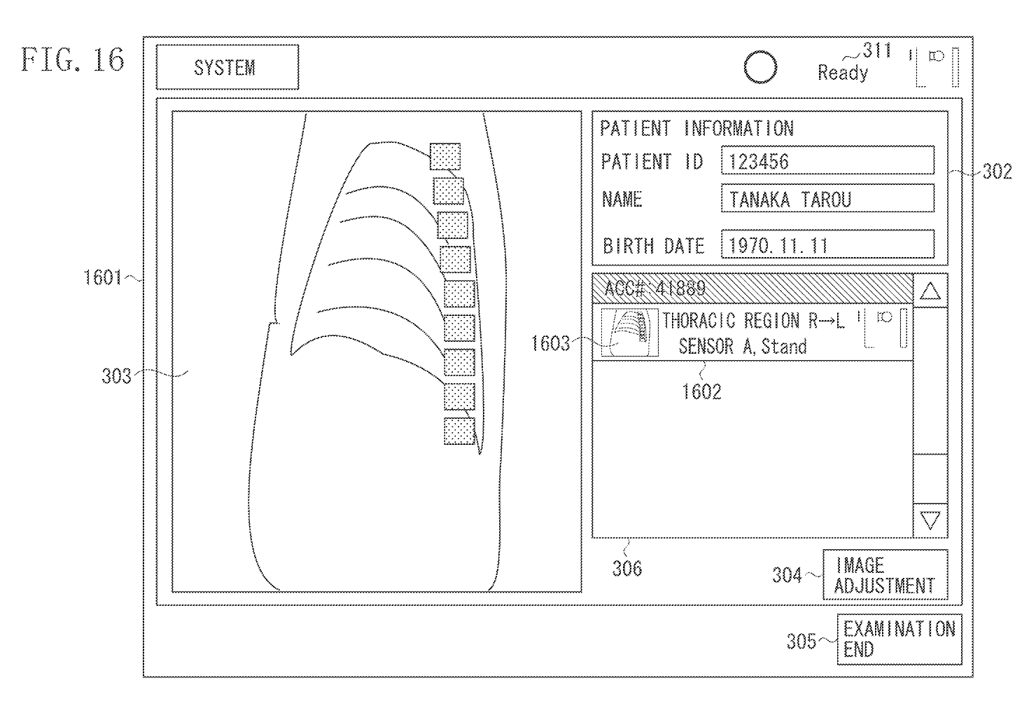

FIGS. 15 and 16 illustrate examples of imaging screens according to the third exemplary embodiment. FIG. 15 illustrates a screen 1501 obtained before the imaging protocol is replaced. FIG. 16 illustrates a screen 1601 obtained after the imaging protocol is replaced. In FIGS. 15 and 16, the components similar to those in FIG. 3 which describes the first exemplary embodiment are given the same reference numerals.

A specific example of a processing flow according to the third exemplary embodiment is described below.

A series of flow from the start to the end of an examination according to the third exemplary embodiment is similar to the one according to the first exemplary embodiment as illustrated in FIG. 8.

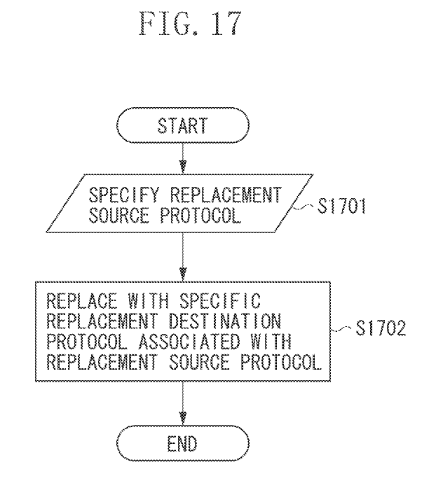

A processing flow for replacing the imaging protocol in step S804 or S807 in FIG. 8 is described below with reference to FIG. 17. It is assumed that the imaging protocol included in the examination order needs to be replaced. For example, in step S806 (S803), if the operator determines that the imaging protocol 1502 of which imaging region is “Thoracic region A→P” needs to be changed to the imaging protocol of which imaging region is “Thoracic region R→L” for any reason, the processing proceeds to step S807 (S804) to perform the replacement of the imaging protocol. A flow of specific processing in step S807 (S804) is illustrated in FIG. 17.

In step S1701, the operator specifies the imaging protocol 1502 as the replacement destination imaging protocol via an operation of the operation unit 114. The display unit 113 may indicate a display for asking the operator's approval as to whether the imaging protocol is replaced. If the operator makes approval via an operation of the operation unit 114, information about the imaging protocol 1502 is input to the replacement source protocol input unit 205. Alternatively, the X-ray imaging control unit may be configured to set whether to indicate a display for asking the operator's approval. If the display for asking the operator's approval is not performed, as soon as the replacement source imaging protocol is specified, the information about the imaging protocol 1502 is input to the replacement source protocol input unit 205. In FIG. 15, the imaging protocol 1502 is displayed as the one already used in imaging, however, the imaging protocol 1502 may be the one not yet used in imaging.

In step S1702, the replacement destination protocol input unit 206 receives the information input to the replacement source protocol input unit 205 to obtain the specific replacement destination imaging protocol associated with the replacement destination imaging protocol from the protocol information storage unit 212. At this point, the operator sets the imaging protocol of which imaging region is “Thoracic region R→L” as a specific replacement destination of the imaging protocol of which imaging region is “Thoracic region A→P” via an operation of the operation unit 114 before the start of examination, and the setting contents are stored in the protocol information storage unit 212. Accordingly, the imaging protocol of which imaging region is “Thoracic region R→L” is input to the replacement destination protocol input unit 206. The protocol replacement unit 209 receives information about the replacement source and replacement destination imaging protocols from the replacement source protocol input unit 205 and the replacement destination protocol input unit 206.

The protocol replacement unit 209 obtains examination information from the examination information storage unit 201 via the examination information obtaining unit 203. The protocol replacement unit 209 replaces the replacement source imaging protocol with the replacement destination imaging protocol in the imaging protocol included in the obtained examination information. Then, the protocol replacement unit 209 updates the examination information stored in the examination information storage unit 201 via the examination information updating unit 204. If the replacement source imaging protocol is not yet used in imaging and ready to be used in imaging, the protocol replacement unit 209 delivers the information about the replacement destination imaging protocol to the imaging control unit 210. The imaging control unit 210 performs the imaging preparation control based on the received replacement destination imaging protocol. If the replacement source imaging protocol is already used in imaging, the protocol replacement unit 209 delivers the information about the replacement destination imaging protocol to the image processing unit 211. The image processing unit 211 performs the image processing again on the image captured by the replacement source imaging protocol (or the image not yet subjected to the image processing) using the image processing parameter associated with the replacement destination imaging protocol.

The display unit 113 switches the display to the examination screen 1601. As illustrated in an imaging protocol 1602 in FIG. 16, the replacement source imaging protocol 1502 is replaced with the imaging protocol of which imaging region is “Thoracic region R→L” as the specific replacement destination. Further, as illustrated in FIG. 16, if the replaced imaging protocol is already used in imaging, the captured image subjected again to the image processing by the image processing unit 211 is displayed in the preview image display area 303 and a thumbnail display area 1603.

According to the third exemplary embodiment, an imaging protocol can be easily replaced with another specific imaging protocol at the time of executing imaging based on the examination order. In addition, the replacement destination is previously determined to allow the operator to save a labor of searching and specifying a replacement destination, and the operator's burden can be reduced.

A fourth exemplary embodiment is described below. The fourth exemplary embodiment mainly assumes an operation required for imaging and diagnosing a pneumoconiosis image in a medical examination.

An X-ray imaging system to which the radiation imaging system according to the fourth exemplary embodiment is applied is similar to the one according to the first exemplary embodiment illustrated in FIG. 1.

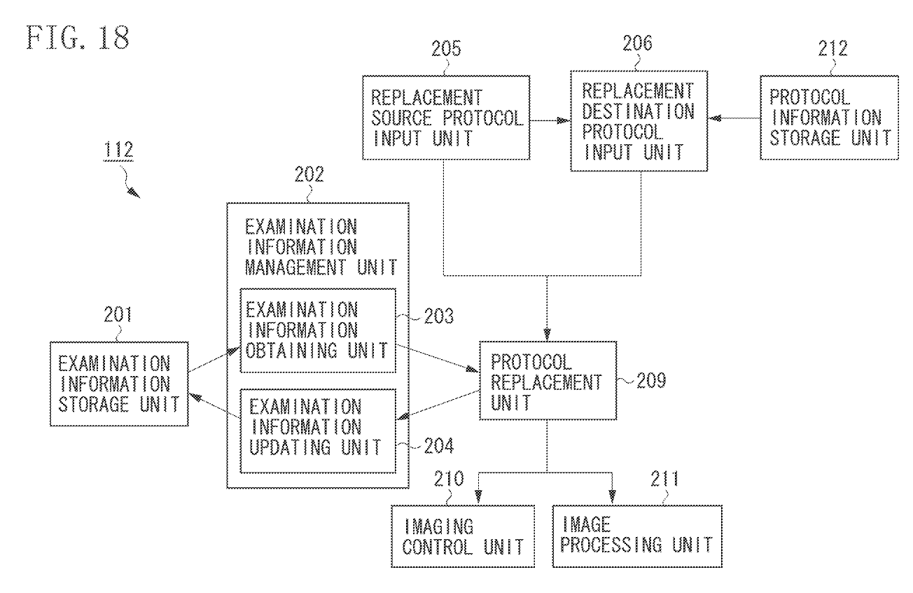

FIG. 18 illustrates an example of a functional configuration for realizing the fourth exemplary embodiment in the X-ray imaging control unit 112 illustrated in FIG. 1. In FIG. 18, the components similar to those in FIG. 2 which describes the first exemplary embodiment are given the same reference numerals.

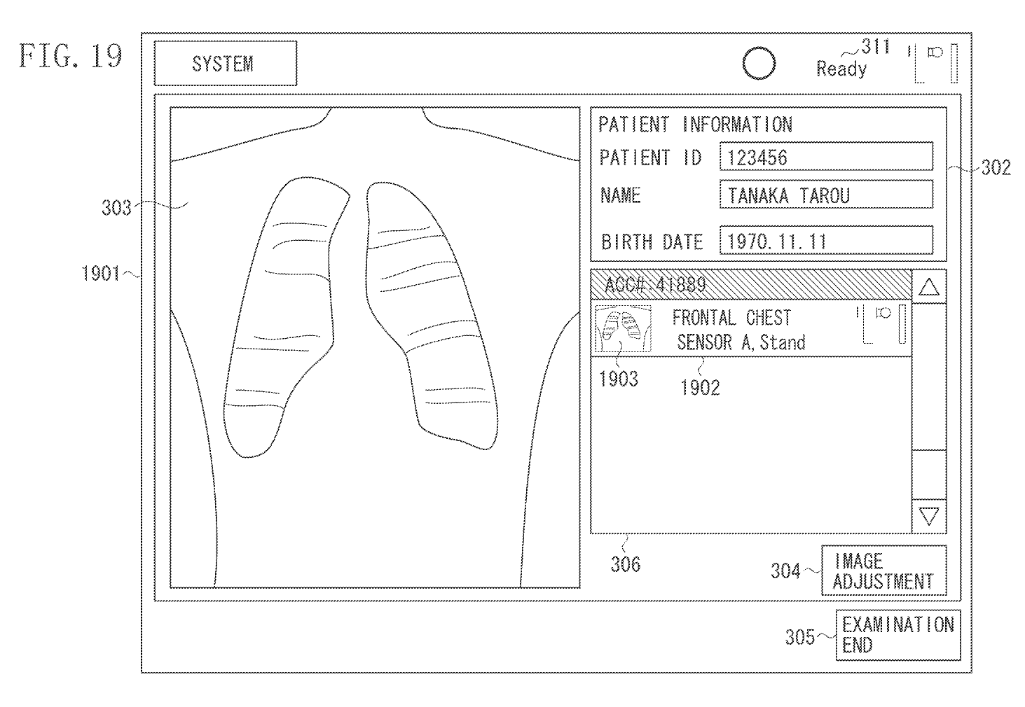

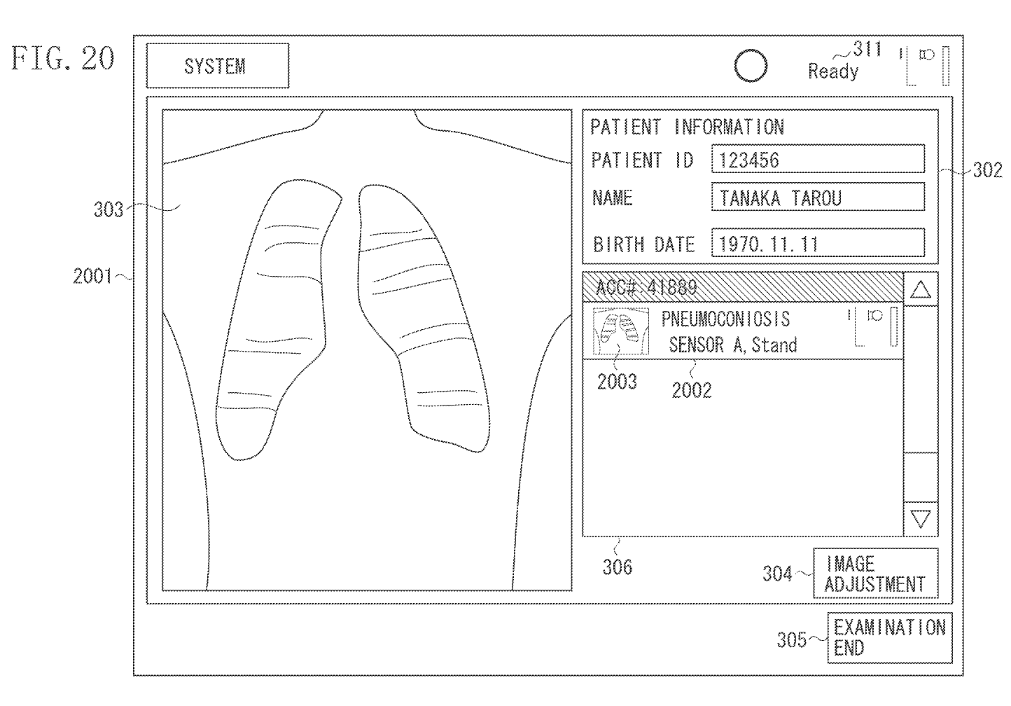

FIGS. 19 and 20 illustrate examples of imaging screens according to the fourth exemplary embodiment. FIG. 19 illustrates an examination screen 1901 in which the imaging protocol whose imaging region is a frontal chest is already used in imaging. FIG. 20 illustrates an examination screen 2001 in which the imaging protocol for pneumoconiosis is already used in imaging. In FIGS. 19 and 20, the components similar to those in FIG. 3 which describes the first exemplary embodiment are given the same reference numerals.

A specific example of a processing flow according to the fourth exemplary embodiment is described below.

A series of flow from the start to the end of an examination according to the fourth exemplary embodiment is similar to the one according to the first exemplary embodiment as illustrated in FIG. 8.

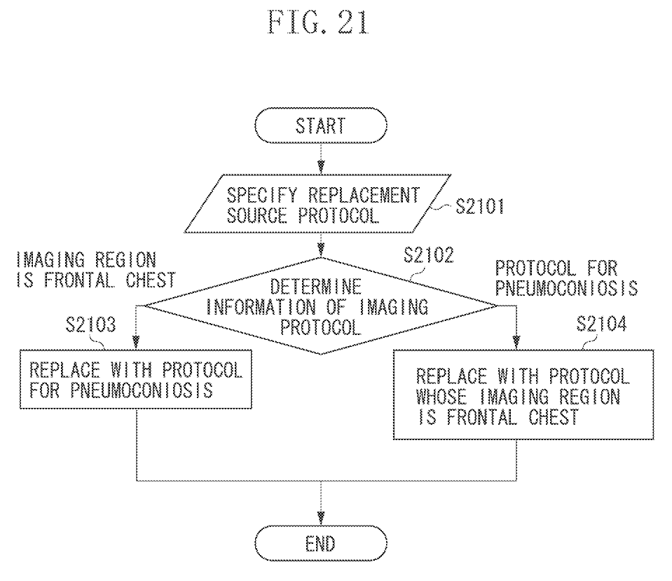

A processing flow for replacing the imaging protocol of which imaging region is a frontal chest is replaced in step S804 or S807 in FIG. 8 is described below with reference to FIG. 21. It is assumed that the imaging protocol included in the examination order needs to be replaced. For example, in step S806 (S803), assuming that an operator determines that an examinee is suspected of having pneumoconiosis based on the captured image of the frontal chest on the examination screen 1901 displayed on the display unit 113. As illustrated in FIG. 19, an imaging protocol 1902 is intended for use in the frontal chest, so that the operator needs to apply the image processing for pneumoconiosis to the captured image. Therefore, the processing proceeds to step S807 (S804) to replace the imaging protocol 1902 for frontal chest with the imaging protocol for pneumoconiosis. A flow of specific processing in step S807 (S804) is illustrated in FIG. 21.

In step S2101, an operator specifies the imaging protocol 1902 as the replacement source imaging protocol via an operation of the operation unit 114. The display unit 113 may indicate a display for asking the operator's approval as to whether the imaging protocol is replaced. If the operator makes approval via an operation of the operation unit 114, information about the imaging protocol 1902 is input to the replacement source protocol input unit 205. Alternatively, the X-ray imaging control unit may be configured to set whether to indicate a display for asking the operator's approval. If the display for asking the operator's approval is not performed, as soon as the replacement source imaging protocol is specified, information about the imaging protocol 1902 is input to the replacement source protocol input unit 205. In FIG. 19, the imaging protocol 1902 is displayed as the one already used in imaging, however, the imaging protocol 1902 may be the one not yet used in imaging. In step S2102, the replacement destination protocol input unit 206 receives the information input to the replacement source protocol input unit 205 to determine information about the replacement source imaging protocol. Since the imaging region of the replacement source imaging protocol is a frontal chest, the processing proceeds to step S2103.

In step S2103, the imaging protocol for pneumoconiosis including the same imaging environment information (“Sensor A, Stand” in this example) as the replacement source imaging protocol 1902 is input to the replacement destination protocol input unit 206. The protocol replacement unit 209 receives information about the replacement source and replacement destination imaging protocols from the replacement source protocol input unit 205 and the replacement destination protocol input unit 206. The protocol replacement unit 209 obtains examination information from the examination information storage unit 201 via the examination information obtaining unit 203. The protocol replacement unit 209 replaces the replacement source imaging protocol with the replacement destination imaging protocol in the imaging protocol included in the obtained examination information. Then, the protocol replacement unit 209 updates the examination information stored in the examination information storage unit 201 via the examination information updating unit 204.

If the replacement source imaging protocol is not yet used in imaging and ready to be used in imaging, the protocol replacement unit 209 delivers the information about the replacement destination imaging protocol to the imaging control unit 210. The imaging control unit 210 performs the imaging preparation control based on the received replacement destination imaging protocol. If the replacement source imaging protocol is already used in imaging, the protocol replacement unit 209 delivers the information about the replacement destination imaging protocol to the image processing unit 211. The image processing unit 211 performs the image processing again on the image captured by the replacement source imaging protocol (or the image not yet subjected to the image processing) using the image processing parameter associated with the replacement destination imaging protocol.

The display unit 113 switches the display to the examination screen 2001. As illustrated in an imaging protocol 2002 in FIG. 20, the replacement source imaging protocol 1902 is replaced with the imaging protocol for pneumoconiosis. Further, as illustrated in FIG. 20, if the replaced imaging protocol is already used in imaging, the captured image subjected again to the image processing by the image processing unit 211 is displayed in the preview image display area 303 and a thumbnail display area 2003.

A processing flow for replacing the imaging protocol for pneumoconiosis in step S804 or S807 in FIG. 8 is described below with reference to FIG. 21. It is assumed that the imaging protocol included in the examination order needs to be replaced. For example, in step S806 (S803), assuming that an operator determines that an examinee is not suspected of having pneumoconiosis based on the captured image on the examination screen 2001 displayed on the display unit 113. As illustrated in FIG. 20, the imaging protocol 2002 is intended for use in the pneumoconiosis, so that the operator needs to apply the image processing for a normal frontal chest to the captured image. Thus, the processing proceeds to step S807 (S804) to replace the imaging protocol 2002 for pneumoconiosis with the imaging protocol of which imaging region is a frontal chest. A flow of specific processing in step S807 (S804) is illustrated in FIG. 21.

In step S2101, the operator specifies the imaging protocol 2002 as the replacement source imaging protocol via an operation of the operation unit 114. The display unit 113 may indicate a display for asking the operator's approval as to whether the imaging protocol is replaced. If the operator makes approval via an operation of the operation unit 114, information about the imaging protocol 2002 is input to the replacement source protocol input unit 205. Alternatively, the X-ray imaging control unit may be configured to set whether to indicate a display for asking the operator's approval. If the display for asking the operator's approval is not performed, as soon as the replacement source imaging protocol is specified, the information about the imaging protocol 2002 is input to the replacement source protocol input unit 205. In FIG. 20, the imaging protocol 2002 is displayed as the one already used in imaging, however, the imaging protocol 2002 may be the one not yet used in imaging. In step S2102, the replacement destination protocol input unit 206 receives the information input to the replacement source protocol input unit 205 to determine information about the replacement source imaging protocol. Since the replacement source imaging protocol is intended for use in pneumoconiosis, the processing proceeds to step S2104.

In step S2104, the imaging protocol of which imaging region is a frontal chest including the same imaging environment information (“Sensor A, Stand” in this example) as the replacement source imaging protocol 2002 is input to the replacement destination protocol input unit 206. The protocol replacement unit 209 receives information about the replacement source and replacement destination imaging protocols from the replacement source protocol input unit 205 and the replacement destination protocol input unit 206. The protocol replacement unit 209 obtains examination information from the examination information storage unit 201 via the examination information obtaining unit 203. The protocol replacement unit 209 replaces the replacement source imaging protocol with the replacement destination imaging protocol in the imaging protocol included in the obtained examination information. Then, the protocol replacement unit 209 updates the examination information stored in the examination information storage unit 201 via the examination information updating unit 204.

If the replacement source imaging protocol is not yet used in imaging and ready to be used in imaging, the protocol replacement unit 209 delivers the information about the replacement destination imaging protocol to the imaging control unit 210. The imaging control unit 210 performs the imaging preparation control based on the received replacement destination imaging protocol. If the replacement source imaging protocol is already used in imaging, the protocol replacement unit 209 delivers the information about the replacement destination imaging protocol to the image processing unit 211. The image processing unit 211 performs the image processing again on the image captured by the replacement source imaging protocol (or the image not yet subjected to the image processing) using the image processing parameter associated with the replacement destination imaging protocol.

Then, the display unit 113 switches the display to the examination screen 1901. As illustrated in the imaging protocol 1902 in FIG. 19, the replacement source imaging protocol 2002 is replaced with the imaging protocol of which imaging region is a frontal chest. Further, as illustrated in FIG. 19, if the replaced imaging protocol is already used in imaging, the captured image subjected again to the image processing by the image processing unit 211 is displayed in the preview image display area 303 and a thumbnail display area 1903.

According to the fourth exemplary embodiment, the imaging protocol of which imaging region is a frontal chest can be easily replaced with the imaging protocol for pneumoconiosis at the time of executing imaging based on the examination order. Inversely, the imaging protocol for pneumoconiosis can be easily replaced with the imaging protocol of which imaging region is a frontal chest to allow reducing the burden of the operator who wants to diagnose an image for pneumoconiosis in a medical examination.

According to one of the exemplary embodiments of the present invention, an imaging protocol based on an examination order can be easily changed at the time of executing imaging.

Accordingly, even if the imaging protocol based on the examination order is desired to be changed at the time of executing imaging, an operator is not burdened with a heavy load.

The present invention can also be realized by executing the following processing. More specifically, software (a program) for realizing the functions of the above exemplary embodiments is supplied to a system or an apparatus via a network or various storage media and a computer (or CPU or micro processing unit (MPU)) of the system or the apparatus reads and executes the program.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2012-086504 filed Apr. 5, 2012, which is hereby incorporated by reference herein in its entirety.

What is claimed is:

1. A radiation imaging system capable of performing imaging based on an examination order including a plurality of imaging protocols, the radiation imaging system comprising:
an imaging unit configured to perform the imaging by a radiation generation unit irradiating an object with a radiation and a radiation detection unit detecting the radiation passing through the object based on the imaging protocols;
an image processing unit configured to perform image processing on the captured image based on the imaging protocols used at the time of imaging by the imaging unit;
a replacement source specification unit configured to specify a replacement source imaging protocol from the examination order based on an instruction from an operator;
a replacement destination specification unit configured to specify one imaging protocol from among a plurality of replacement destination imaging protocols obtained based on information attached to the imaging protocol specified by the replacement source specification unit; and
a replacement unit configured to replace the imaging protocol specified by the replacement source specification unit with the imaging protocol specified by the replacement destination specification unit.

2. The radiation imaging system according to claim 1,
wherein the replacement destination specification unit includes:
an extraction unit configured to extract a candidate for a replacement destination imaging protocol based on information attached to the imaging protocol specified by the replacement source specification unit; and
a display unit configured to display a list of the imaging protocols extracted by the extraction unit under a desired condition, and
the replacement destination specification unit specifies a replacement destination protocol from the list of the imaging protocols displayed by the display unit based on an instruction from an operator.

3. The radiation imaging system according to claim 2,
wherein, if the imaging protocol specified by the replacement source specification unit is not yet used in imaging, the extraction unit extracts all the imaging protocols registered in the radiation imaging system as candidates for the replacement destination protocol and,
if the imaging protocol specified by the replacement source specification unit is already used in imaging, the extraction unit extracts only the imaging protocol associated with imaging environment information same as the imaging protocol specified by the replacement source specification unit among the imaging protocols registered in the radiation imaging system.

4. The radiation imaging system according to claim 2,
wherein the display unit preferentially displays a candidate for a replacement destination protocol of an imaging region in the vicinity of the imaging protocol specified by the replacement source specification unit.

5. The radiation imaging system according to claim 2,
wherein the display unit preferentially displays an imaging protocol previously specified by the radiation imaging system.

6. The radiation imaging system according to claim 2,
wherein the display unit highlights an imaging protocol specified by the replacement source specification unit among candidates for the replacement destination imaging protocols displayed in the list and a replacement destination imaging protocol specified by an instruction from an operator.

7. The radiation imaging system according to claim 1,
wherein the replacement destination specification unit includes a specific replacement destination automatic specification unit configured to automatically specify a specific imaging protocol as a replacement destination imaging protocol based on information attached to the imaging protocol specified by the replacement source specification unit.

8. The radiation imaging system according to claim 7,
wherein the imaging protocol includes information about the specific replacement destination imaging protocol associated with imaging environment information same as the imaging environment information associated with the imaging protocol itself and
the specific replacement destination specification unit automatically specifies the specific replacement destination imaging protocol associated with the imaging protocol specified by the replacement source specification unit as the replacement destination imaging protocol.

9. The radiation imaging system according to claim 1,
wherein, if an imaging region of an imaging protocol specified by the replacement source specification unit is a frontal chest, the specific replacement destination automatic specification unit automatically specifies an imaging protocol for pneumoconiosis associated with imaging environment information same as the imaging protocol specified by the replacement source specification unit as the replacement destination imaging protocol, and
if an imaging protocol specified by the replacement source specification unit is an imaging protocol for pneumoconiosis, the specific replacement destination automatic specification unit automatically specifies an imaging protocol of which imaging region is a frontal chest and which is associated with the same imaging environment information as the imaging protocol specified by the replacement source specification unit as the replacement destination imaging protocol.

10. The radiation imaging system according to claim 1, further comprising an image processing unit configured to process a radiation image associated with the imaging information according to a change based on the changed imaging region.

11. The radiation imaging system according to claim 1,
wherein the imaging protocol includes at least any one piece of information about the radiation generation unit, image processing of the image processing unit, the imaging environment, and the imaging region.

12. The radiation imaging system according to claim 1,
wherein the imaging environment information includes at least any one piece of information about the radiation generation unit and an imaging orientation.

13. A control apparatus for radiation imaging capable of changing radiation imaging information, the control apparatus comprising:
a specification unit configured to specify at least one of a plurality of imaging information pieces including radiation imaging information planned to be used in imaging and imaging information associated with a captured radiation image;
a display control unit configured to display at least one correction candidate for an imaging region included in the imaging information according to whether the specified imaging information is the radiation imaging information planned to be used or the imaging information associated with the captured radiation image;
a change unit configured to, according to an instruction to select at least one of the displayed correction candidates, change information about the imaging region included in the imaging information to the selected correction candidate; and
an image processing unit configured to process a radiation image associated with the imaging information according to the change based on the changed imaging region.

14. A control apparatus for radiation imaging, the control apparatus comprising:
an obtaining unit configured to obtain imaging information for performing radiation imaging;
a display control unit configured to display a display screen for displaying the imaging information and a radiation image captured based on the imaging information; and
a change destination obtaining unit configured to, with respect to one imaging information displayed on the display control unit, obtain at least one change destination for the displayed imaging information, based on the imaging information; and,
a control unit configured to change the one imaging information displayed on the display control unit to a change destination designated from pieces of the obtained change destination.

15. The control apparatus according to claim 14, further comprising:
a change source specification unit configured to specifies the one imaging information about a change source according to an operation input to an operation unit; and
a change destination specification unit configured to specifies imaging information about a change destination according to an operation input to the operation unit.

16. The control apparatus according to claim 14,
wherein the change destination obtaining unit obtains the imaging information about the change destination based on whether a radiation image obtained by radiation imaging based on the one imaging information exists.

17. The control apparatus according to claim 16,
wherein, if the radiation image obtained by the radiation imaging based on the one imaging information exists, the change destination obtaining unit obtains the imaging information about the change destination from imaging information corresponding to a radiation imaging apparatus to be used for the radiation imaging based on the one imaging information.

18. The control apparatus according to claim 16,
wherein, if the radiation image obtained by the radiation imaging based on the one imaging information does not exist, the change destination obtaining unit obtains the imaging information about the change destination from imaging information corresponding to an imaging technique included in the one imaging information.

19. A method for controlling a radiation imaging system capable of performing imaging based on an examination order including a plurality of imaging protocols, the method comprising:
performing the imaging by a radiation generation unit irradiating an object with a radiation and a radiation detection unit detecting the radiation passing through the object based on imaging protocols;
performing image processing on the captured image based on the imaging protocols used at the time of imaging by the imaging unit;
specifying a replacement source imaging protocol from the examination order based on an instruction from an operator;
specifying one imaging protocol from among a plurality of replacement destination imaging protocols obtained based on information attached to the imaging protocol specified by a source specification unit; and
replacing the imaging protocol specified by the replacement source specification unit with an imaging protocol specified by a replacement destination specification unit.

20. A method for controlling radiation imaging capable of changing radiation imaging information, the method comprising:
specifying at least one of a plurality of imaging information pieces including radiation imaging information planned to be used in imaging and imaging information associated with a captured radiation image;
displaying a correction candidate for an imaging region included in the imaging information according to whether the specified imaging information is the radiation imaging information planned to be used or the imaging information associated with the captured radiation image;
changing, according to an instruction to select at least one of the displayed correction candidates, information about the imaging region included in the imaging information to the selected correction candidate; and
processing a radiation image associated with the imaging information according to the change based on the changed imaging region.

21. A method for controlling radiation imaging, the method comprising:
obtaining imaging information for performing radiation imaging;
displaying a display screen for displaying the imaging information and a radiation image captured based on the imaging information; and
specifying one imaging information displayed on the display screen;
obtaining candidates for imaging information about at least one change destination based on the one specified imaging information;
specifying imaging information about the change destination from among the candidates; and
changing a display of the one specified imaging information on the display screen to the imaging information of the specified change destination.

22. A non-transitory computer-readable storage medium storing a method according to claim 19.

23. A non-transitory computer-readable storage medium storing a method according to claim 20.

24. A non-transitory computer-readable storage medium for storing the method according to claim 21.
